(19) United States
(12) Patent Application Publication
(10) Pub. No.: US 2026/0234090 A1
(43) Pub. Date: Aug. 13, 2026

(54) AZEOTROPIC EXTRACTIVE DISTILLATION FOR ENHANCED METHANOL REFINEMENT

(71) Applicant: Lummus Technology LLC, Houston, TX (US)

(72) Inventors: Rosette Barias, Houston, TX (US); Michael Jon Scott, Houston, TX (US)

(73) Assignee: Lummus Technology LLC, Houston, TX (US)

(21) Appl. No.: 19/535,883

(22) Filed: Feb. 10, 2026

Related U.S. Application Data
(60) Provisional application No. 63/756,690, filed on Feb. 10, 2025.

(51) Int. Cl. C07C 29/82; B01D 3/14; B01D 3/36; B01D 3/40

(52) U.S. Cl. CPC C07C 29/82; B01D 3/141; B01D 3/143; B01D 3/36; B01D 3/40

(57) ABSTRACT

Systems and processes for purifying a crude methanol stream including obtaining a crude methanol feedstock comprising methanol, water, acetone, methyl formate, and optionally one or more of ethanol, propanol, and butanol. The crude methanol is contacted with a glycol, a glycerol, or a mixture of glycols in an azeotropic distillation column to recover a first overhead stream comprising the acetone and methyl formate and a first bottoms stream comprising the glycol or glycerol, water, methanol, and, when contained in the feed, the ethanol, propanol and butanol. The first bottoms stream is then separated, in some embodiments, in a dividing wall distillation column to recover a second overhead stream comprising methanol and water, a second bottoms stream comprising the glycol or glycerol, a side draw stream comprising water and, when contained in the feed, the ethanol, propanol and butanol.

### BACKGROUND

Processes for production of methanol include, for example, gasification of biomass or coal or synthesis from natural gas. Said processes produce what will be referred to herein as “crude methanol,” which may be sold or used as a raw material to produce other chemicals. The crude methanol may include methanol and several impurities, such as water, formaldehyde, acetone, etc.

A highly purified methanol product may be advantageously used to produce a higher purity chemical products. Accordingly, there exists a need for systems and methods for producing high purity methanol which may then be used to synthesize additional high purity chemical products.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a process for purifying a methanol stream, including obtaining a crude methanol feedstock including methanol, water, acetone, methyl formate, and optionally one or more of ethanol, propanol, and butanol, contacting the crude methanol with a glycol, a glycerol, or a mixture of glycols in an azeotropic distillation column to recover a first overhead stream including the acetone and methyl formate and a first bottoms stream including the glycol or glycerol, water, methanol, and, when contained in the feed, the ethanol, propanol and butanol, and separating the first bottoms stream in a dividing wall distillation column to recover a second overhead stream including methanol and water, a second bottoms stream including the glycol or glycerol, a side draw stream including water and, when contained in the feed, the ethanol, propanol and butanol.

In another aspect, embodiments disclosed herein relate to a process for purifying a methanol stream, including obtaining a crude methanol feedstock including methanol, water, acetone, methyl formate, and optionally one or more of ethanol, propanol, and butanol, contacting the crude methanol with a glycol, a glycerol, or a mixture of glycols in an azeotropic distillation column to recover a first overhead stream including the acetone and methyl formate and a first bottoms stream including the glycol or glycerol, water, methanol, and, when contained in the feed, the ethanol, propanol and butanol, and separating the first bottoms stream in a methanol tailing column to recover a third overhead stream including methanol and water, a third bottoms stream including the glycol or glycerol, water and, when contained in the feed, the ethanol, propanol and butanol.

In yet another aspect, embodiments disclosed herein relate to A system for purifying a methanol stream, including a feed stream for supplying a crude methanol feedstock including methanol, water, one or more hydrocarbon impurities that form an azeotrope with methanol and one or more hydrocarbons having a boiling point greater than methanol, an azeotropic distillation column configured for contacting the crude methanol with a glycol or glycerol to recover a first overhead stream including the one or more hydrocarbon impurities that form an azeotrope with methanol and a first bottoms stream including the glycol or glycerol, water, methanol, and the one or more hydrocarbons having a boiling point greater than methanol, and a dividing wall distillation column configured for separating the first bottoms stream to recover a second overheads stream including methanol and water, a second bottoms stream including the glycol or glycerol, and a side draw stream including water and the one or more hydrocarbons having a boiling point greater than methanol.

Other aspects and advantages of the claimed subject matter will be apparent from the following description and the appended claims.

### BRIEF DESCRIPTION OF DRAWINGS

FIG. 1 is a methanol purification system according to one or more embodiments.

FIG. 2 is a second methanol purification system according to one or more embodiments.

### DETAILED DESCRIPTION

Typically, methanol purification uses conventional distillation columns that rely on separating methanol from impurities by simple distillation, often requiring a series of columns to achieve the desired purity, especially when dealing with low-purity or waste-derived methanol sources or with impurities that form an azeotrope with methanol.

Embodiments disclosed herein generally relate to systems and processes for methanol purification. In some embodiments, purified methanol produced from systems and processes disclosed herein may be sold or used as a feed in a process to produce high purity additional chemicals. The additional chemicals may include but are not limited to formaldehyde, acetic acid, methyl tertiary-butyl ether (MTBE), methyl methacrylate, olefins (such as ethylene and propylene), dimethyl ether (DME), and gasoline additives.

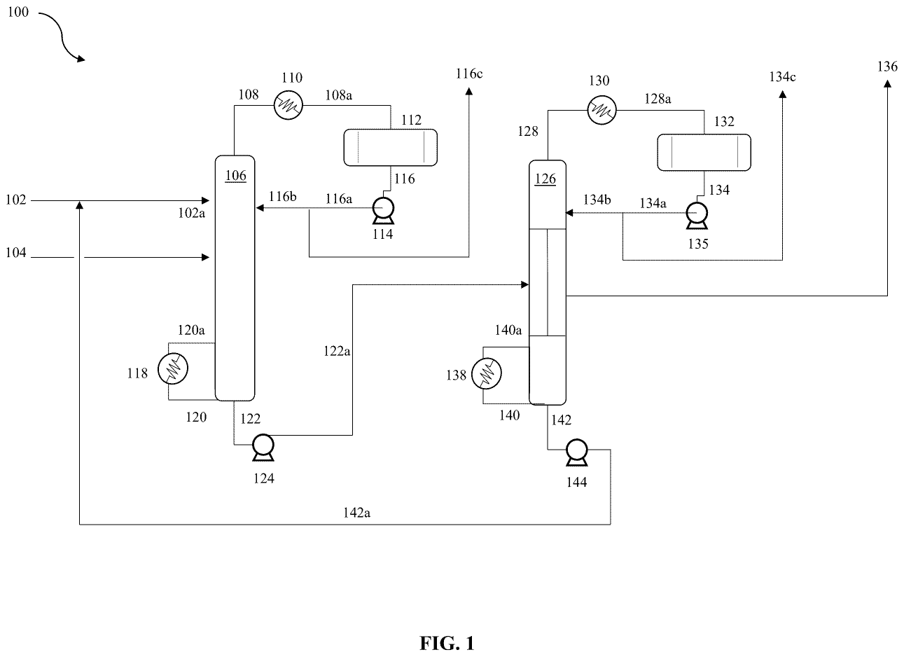

Turning to FIG. 1, FIG. 1 is a process flow diagram of a methanol purification system 100 according to one or more embodiments. The methanol purification system 100 may include an azeotropic distillation column 106 configured to receive a crude methanol feedstock 104 and a glycol or glycerol feed 102. The methanol purification system 100 may also include a dividing wall distillation column 126 configured to receive a first bottoms stream 122 and produce a purified methanol product 134c, a second bottoms stream 142, and a side draw stream 136. While not explicitly shown, systems and processes disclosed herein may include additional elements such as pumps, valves, vessels, storage tanks, and other equipment commonly used for the processes described, as would be understood by one of ordinary skill in the art.

In the methanol purification system 100, the crude methanol feedstock 104 and the glycol or glycerol feed enter the azeotropic distillation column 106. In some embodiments, the glycol or glycerol feed 102 may be mixed with a glycol or glycerol recycle 142a to produce a combined glycol or glycerol feed 102a. The glycol or glycerol recycle 142a may be produced by one or more downstream processes, as will be described in more detail below. The glycol or glycerol feed 102 may primarily contain any suitable form of glycol or glycerol, such as ethylene or propylene glycol, or glycerol as a non-limiting example, where the glycol or glycerol or the mixture of glycols has a boiling point greater than that of water.

The crude methanol feedstock 104 of one or more embodiments may contain a number of components, including but not limited to methanol, water, one or more hydrocarbon impurities that form an azeotrope with methanol, and one or more hydrocarbons having a boiling point greater than the boiling point of methanol. In some embodiments, the crude methanol feedstock 104 contains methanol, water, methyl formate, acetone, and one or more of ethanol, propanol, and butanol. As would be understood by one of ordinary skill in the art, other chemical components/contaminants may be present in the crude methanol feedstock 104 depending on the source and prior processing (if applicable) of the crude methanol feedstock 104. The crude methanol feedstock 104 may be obtained from any suitable source known in the art. In some embodiments, the crude methanol feedstock 104 is obtained from a municipal waste-derived or biomass-derived methanol production processes. Examples of biomass-derived methanol sources include but are not limited to agricultural residues, woody biomass, and black liquor.

The crude methanol feedstock 104 and the glycol or glycerol feed 102 (or the combined glycol or glycerol feed 102a) may be contacted in the azeotropic distillation column 106 where the feeds may be distilled to recover a first overhead stream 108 and a first bottoms stream 122. The first overhead stream 108 includes the one or more hydrocarbon impurities that form an azeotrope with methanol that were present in the crude methanol feedstock 104 (such as, for example, acetone). Contacting the glycol or glycerol feed 102 (or the combined glycol or glycerol feed 102a) with the crude methanol feedstock 104 may advantageously help facilitate separation of impurities from the crude methanol feedstock 104 through distillation by breaking the azeotrope between the impurities which form an azeotrope with methanol (such as acetone) and the methanol.

The azeotropic distillation column 106 may be any suitable azeotropic distillation column system known in the art. The azeotropic distillation column 106 may be operated at any suitable conditions (combinations of pressure and temperature, feed rate or feed ratio of glycol or glycerol, etc.) to facilitate the desired separations.

In some embodiments, the first overhead stream 108 may be cooled via a first heat exchanger 110 (e.g., an overhead condenser) to produce a first condensed overhead stream 108a. The first condensed overhead stream 108a may then enter a first reflux drum 112 to produce a first distillate stream 116. Non-condensed vapors, if any, may be recovered via a vapor outlet (not shown) from the reflux drum. The first distillate stream 116 may then be pumped by a first pump 114 to produce a first pressurized distillate stream 116a. A first portion of the first pressurized distillate stream 116a may be fed as a reflux stream 116b introduced to an upper portion of the azeotropic distillation column 106. A second portion of the first pressurized distillate stream 116a may be withdrawn as a light impurities stream 116c. The light impurities stream 116c includes the one or more hydrocarbon impurities that form an azeotrope with methanol that were present in the crude methanol feedstock 104 (such as, for example, acetone).

The first bottoms stream 122 includes the glycol or glycerol, the methanol, the water, and one or more hydrocarbons having a boiling point greater than the boiling point of methanol that were included in the crude methanol feedstock 104 (such as, for example, ethanol, propanol, and/or butanol).

In some embodiments, a first bottoms draw 120 (or a portion of first bottoms stream 122) may be heated via a second heat exchanger 118 (e.g., a reboiler) to produce a first reboil stream 120a. The first reboil stream 120a may be fed back to the azeotropic distillation column 106 as shown in FIG. 1, providing heat to drive the desired separations.

In some embodiments, the first bottoms stream 122 may be pumped via a second pump 124 to produce a first pressurized bottoms stream 122a. The first bottoms stream 122 (or the first pressurized bottoms stream 122a) may then be sent to a dividing wall distillation column 126. In the dividing wall distillation column 126, the first bottoms stream 122 (or the first pressurized bottoms stream 122a) is separated to produce a second overhead stream 128, a second bottoms stream 142, and a side draw stream 136.

The dividing wall distillation column 126 may be any suitable dividing wall distillation column system known in the art. In some embodiments, the dividing wall distillation column includes a mid-wall, with a common rectification section and a common stripping section located above and below the mid-wall, respectively. Other types of dividing wall distillation columns known in the art may also be used (top wall with separate overhead systems, for example). The dividing wall distillation column 126 may be operated at any suitable conditions (pressure, temperature, reflux rate or reflux ratio, etc.) to achieve the desired separations.

The second overhead stream 128 of one or more embodiments includes methanol and water. In some embodiments, the second overhead stream 128 may be cooled via third heat exchanger 130 (e.g., an overhead condenser) to produce a second condensed overhead stream 128a. In some embodiments, the second condensed overhead stream 128a may be sent to a second reflux drum 132 to produce a second distillate stream 134. Non-condensed vapors, if any, may be recovered via a vapor outlet (not shown) from the reflux drum. The second distillate stream 134 may then be pumped by a third pump 135 to produce a second pressurized distillate stream 134a. A first portion of the second pressurized distillate stream 134a may be fed as a reflux stream 134b introduced to an upper portion of the dividing wall distillation column 126. A second portion of the second pressurized distillate stream 134a may be withdrawn as purified methanol product 134c. The purified methanol product 134c may include primarily methanol and a minute amount of water. In some embodiments, the purified methanol product 134c may have a methanol purity of at least 95 wt %, at least 98 wt. %, at least 99 wt %, or at least 99.5 wt %.

The second bottoms stream 142 of one or more embodiments includes glycol or glycerol. Other various heavy impurities as contained in the crude methanol feedstock may also be present, buildup of which may be prevented by removing a portion (not shown) of the second bottoms stream 142 from the recirculation loop. In some embodiments, the second bottoms stream 142 may be pressurized via fourth pump 144 to produce a glycol or glycerol recycle 142a. As described above, the glycol or glycerol recycle 142a may mix with the glycol or glycerol feed 102 (or mixed with a make-up glycol or glycerol feed as needed) to produce a combined glycol or glycerol feed 102a.

The side draw stream 136 of one or more embodiments includes water and the one or more hydrocarbons having a boiling point greater than methanol (such as, for example, ethanol, propanol, and/or butanol).

In some embodiments, a second bottoms draw 140 (or a portion of second bottoms stream 142) may be recovered from the dividing wall distillation column 126 and heated via a fourth heat exchanger 138 (e.g., a reboiler) to produce a second reboil stream 140a. The second reboil stream 140a may be fed back to the dividing wall distillation column 126 as shown in FIG. 1, providing heat to drive the desired separations within the column.

In some embodiments, the purified methanol product 134c may be collected and sold as a final product. In some embodiments, the purified methanol product 134c may be subjected to further processing. As a non-limiting example, the purified methanol product 134c may be sent to a reaction system to be converted to additional chemicals, or sold as-is.

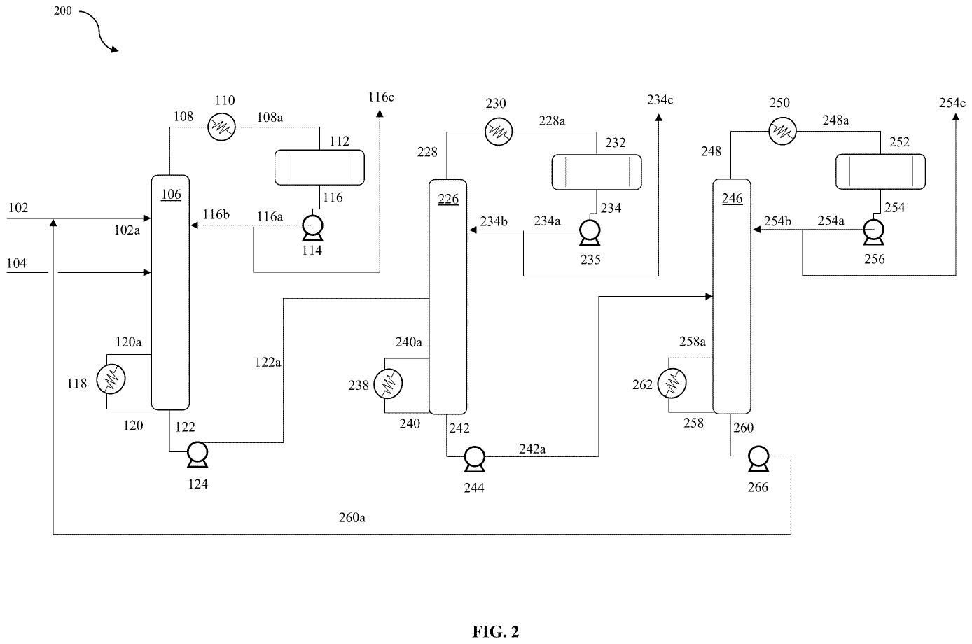

Turning to FIG. 2, FIG. 2 is a process flow diagram of a second methanol purification system 200 according to one or more embodiments. The second methanol purification system 200 may include an azeotropic distillation column 106 configured to receive a crude methanol feedstock 104 and a glycol or glycerol feed 102. The second methanol purification system 200 may also include a methanol tailing column 226 configured to receive a first pressurized bottoms stream 122a and produce a second purified methanol product 234c and a third pressurized bottoms stream 242a, and a solvent fractionator system configured to receive the third pressurized bottoms stream 242a and produce a second glycol or glycerol recycle 260a and a heavy impurities fraction 254c. While not explicitly shown, systems and processes disclosed herein may include additional elements such as pumps, valves, vessels, storage tanks, and other equipment commonly used for the processes described, as would be understood by one of ordinary skill in the art.

In the second methanol purification system 200, the crude methanol feedstock 104 and the glycol or glycerol feed enter the azeotropic distillation column 106. In some embodiments, the glycol or glycerol feed 102 (makeup glycol or glycerol, fed as needed) may be mixed with the second glycol or glycerol recycle 260a to produce a combined glycol or glycerol feed 102a. The second glycol or glycerol recycle 260a may be produced by one or more downstream processes, as will be described in more detail below. The glycol or glycerol feed 102 may primarily contain any suitable form of glycol or glycerol, such as ethylene glycol or glycerol, as a non-limiting example, where the glycol or glycerol or the mixture of glycols has a boiling point greater than that of water.

The crude methanol feedstock 104 of one or more embodiments may contain a number of components, including but not limited to methanol, water, one or more hydrocarbon impurities that form an azeotrope with methanol, and one or more hydrocarbons having a boiling point greater than the boiling point of methanol. In some embodiments, the crude methanol feedstock 104 contains methanol, water, methyl formate, acetone, and one or more of ethanol, propanol, and butanol. As would be understood by one of ordinary skill in the art, other chemical components/contaminants may be present in the crude methanol feedstock 104 depending on the source and prior processing (if applicable) of the crude methanol feedstock 104. The crude methanol feedstock 104 may be obtained from any suitable source known in the art. In some embodiments, the crude methanol feedstock 104 is obtained from a municipal waste-derived or biomass-derived methanol production process.

The crude methanol feedstock 104 and the glycol or glycerol feed 102 (or the combined glycol or glycerol feed 102a) may be contacted in the azeotropic distillation column 106 where the feeds may be distilled to recover a first overhead stream 108 and a first bottoms stream 122. The first overhead stream 108 includes the one or more hydrocarbon impurities that form an azeotrope with methanol that were present in the crude methanol feedstock 104 (such as, for example, acetone). Contacting the glycol or glycerol feed 102 (or the combined glycol or glycerol feed 102a) with the crude methanol feedstock 104 may advantageously help facilitate separation of impurities from the crude methanol feedstock 104 through distillation by breaking the azeotrope between the impurities which form an azeotrope with methanol (such as acetone) and the methanol.

The azeotropic distillation column 106 may be any suitable azeotropic distillation column system known in the art. The azeotropic distillation column 106 may be operated at any suitable conditions (combinations of pressure and temperature, feed rate or feed ratio of glycol or glycerol, etc.) to facilitate the desired separations.

In some embodiments, the first overhead stream 108 may be cooled via a first heat exchanger 110 (e.g., an overhead condenser) to produce a first condensed overhead stream 108a. The first condensed overhead stream 108a may then enter a first reflux drum 112 to produce a first distillate stream 116. Non-condensed vapors, if any, may be recovered via a vapor outlet (not shown) from the reflux drum. The first distillate stream 116 may then be pumped by a first pump 114 to produce a first pressurized distillate stream 116a. A first portion of the first pressurized distillate stream 116a may be fed as a reflux stream 116b introduced to an upper portion of the azeotropic distillation column 106. A second portion of the first pressurized distillate stream 116a may be withdrawn as a light impurities stream 116c. The light impurities stream 116c includes the one or more hydrocarbon impurities that form an azeotrope with methanol that were present in the crude methanol feedstock 104 (such as, for example, acetone).

The first bottoms stream 122 includes the glycol or glycerol, the methanol, the water, and the one or more hydrocarbons having a boiling point greater than the boiling point of methanol that were included in the crude methanol feedstock 104 (such as, for example, ethanol, propanol, and/or butanol).

In some embodiments, a first bottoms draw 120 (or a portion of first bottoms stream 122) may be heated via a second heat exchanger 118 (e.g., a reboiler) to produce a first reboil stream 120a. The first reboil stream 120a may be fed back to the azeotropic distillation column 106 as shown in FIG. 2, providing heat to drive the desired separations.

In some embodiments, the first bottoms stream 122 may be pumped via a second pump 124 to produce a first pressurized bottoms stream 122a. The first bottoms stream 122 (or the first pressurized bottoms stream 122a) may then be sent to a methanol tailing column 226. In the methanol tailing column 226, the first bottoms stream 122 (or the first pressurized bottoms stream 122a) is separated to produce a third overhead stream 228 and a third bottoms stream 242.

The methanol tailing column 226 may be operated at any suitable conditions (temperature, pressure, reflux ratio, etc.) to achieve the desired separations.

The third overhead stream 228 of one or more embodiments includes methanol and water. In some embodiments, the third overhead stream 228 may be cooled via a fifth heat exchanger 230 to produce a third condensed overhead stream 228a. In some embodiments, the third condensed overhead stream 228a may be sent to a third reflux drum 232 to produce a third distillate stream 234. Non-condensed vapors, if any, may be recovered via a vapor outlet (not shown) from the reflux drum. The third distillate stream 234 may then be pumped by a fifth pump 235 to produce a third pressurized distillate stream 234a. A first portion of the third pressurized distillate stream 234a may be fed as a reflux stream 234b introduced to an upper portion of the methanol tailing column 226. A second portion of the third pressurized distillate stream 234a may be withdrawn as second purified methanol product 234c. The second purified methanol product 234c may include primarily methanol and a minute amount of water. In some embodiments, the second purified methanol product 234c may have a methanol purity of at least 95 wt %, at least 98 wt. %, at least 99 wt %, or at least 99.5 wt %.

In some embodiments, a third bottoms draw 240 (or a portion of third bottoms stream 242) may be recovered from the methanol tailing column 226 and heated via a sixth heat exchanger 238 (e.g., a reboiler) to produce a third reboil stream 240a. The third reboil stream 240a may be fed back to the methanol tailing column 226 as shown in FIG. 2, providing heat to drive the desired separations within the column.

The third bottoms stream 242 of one or more embodiments may include water, one or more hydrocarbons having a boiling point greater than methanol (such as, for example, ethanol, propanol, and/or butanol introduced with the crude methanol feed), and glycol or glycerol. In some embodiments, the third bottoms stream 242 may be pressurized via sixth pump 244 to produce a third pressurized bottoms stream 242a. The third pressurized bottoms stream 242a may then be fed to a solvent fractionator column 246. In the solvent fractionator column 246, the third bottoms stream 242 (or the third pressurized bottoms stream 242a) is separated to produce a fourth overhead stream 248 and a fourth bottoms stream 260.

The solvent fractionator column 246 may be any suitable solvent fractionator column system known in the art. The solvent fractionator column may be operated at any suitable conditions (combinations of pressure and temperature, feed rate, reflux ratio, etc.) to facilitate the desired separations.

The fourth overhead stream 248 of one or more embodiments includes water and the one or more hydrocarbons having a boiling point greater than methanol (such as, for example, ethanol, propanol, and/or butanol). In some embodiments, the fourth overhead stream 248 may be cooled via a seventh heat exchanger 250 (e.g., a condenser) to produce a fourth condensed overhead stream 248a. In some embodiments, the fourth condensed overhead stream 248a may be sent to a fourth reflux drum 252 to produce a fourth distillate stream 254. Non-condensed vapors, if any, may be recovered via a vapor outlet (not shown) from the reflux drum. The fourth distillate stream 254 may then be pumped by a seventh pump 256 to produce a fourth pressurized distillate stream 254a. A first portion of the fourth pressurized distillate stream 254a may be fed as a reflux stream 254b introduced to an upper portion of to the solvent fractionator column 246. A second portion of the fourth pressurized distillate stream 254a may be withdrawn as a heavy impurities fraction 254c. The heavy impurities fraction includes water and the one or more hydrocarbons having a boiling point greater than methanol (such as, for example, ethanol, propanol, and/or butanol).

In some embodiments, the second purified methanol product 234c may be collected and sold as a final product. In some embodiments, the second purified methanol product 234c may be subjected to further processing. As a non-limiting example, the second purified methanol product 234c may be sent to a reaction system to be converted to additional chemicals.

In some embodiments, a fourth bottoms draw 258 (or a portion of fourth bottoms stream 260) may be recovered from the solvent fractionator column 246 and heated via an eighth heat exchanger 262 (e.g., a reboiler) to produce a fourth reboil stream 258a. The fourth reboil stream 258a may be fed back to the solvent fractionator column 246 as shown in FIG. 2, providing heat to drive the desired separations within the column.

The fourth bottoms stream 260 of one or more embodiments may include primarily glycol or glycerol. Other various heavy impurities as contained in the crude methanol feedstock may also be present, buildup of which may be prevented by removing a portion (not shown) of the fourth bottoms stream 260 from the recirculation loop. In some embodiments, the fourth bottoms stream 260 may be pressurized via eighth pump 266 to produce a second glycol or glycerol recycle 260a. As described above, the second glycol or glycerol recycle 260a may mix with the glycol or glycerol feed 102 (or mixed with a make-up glycol or glycerol feed as needed) to produce a combined glycol or glycerol feed 102a.

Embodiments disclosed herein may include at least one of the following advantages.

Systems and processes for methanol purification according to embodiments disclosed herein may advantageously combine dividing wall column technology and azeotropic distillation, leading to a simplified process with fewer steps and reduced operational complexity. Dividing wall column technology and azeotropic distillation processes are conventionally treated as separate tools for improving distillation efficiency. As such, combining the aforementioned technologies in a glycol or glycerol-based azeotropic dividing wall column for acetone removal according to embodiments disclosed herein would not have been typically considered due to the specialized functions of each technology. Dividing wall column technology and azeotropic distillation processes according to embodiments disclosed herein produce an unexpected synergy not present in conventional design processes. In contrast, prior methods often require more intricate setups with multiple columns and/or solvent recovery stages.

Embodiments disclosed herein effectively produce high-purity methanol by separating out impurities like acetone, methyl formate, and heavier alcohols, while enabling glycol or glycerol recovery and recycling, leading to improved cost efficiency and sustainability. The use of glycol or glycerol advantageously acts as a solvent to break azeotropes of methanol with acetone, making the acetone separation from methanol easier.

Embodiments disclosed herein may advantageously use highly impure feedstocks, such as municipal waste, to produce high value chemical products. There is a growing demand worldwide for renewable energy sources and waste-to-energy technologies. As such, utilizing waste-derived or biomass derived methanol to produce valuable chemicals using systems and processes according to one or more embodiments disclosed herein promotes sustainability and offers a solution for recycling waste into a useful fuel.

Systems and processes disclosed in one or more embodiments herein include using extractive distillation with glycol or glycerol to remove lighter impurities such as acetone and a dividing wall column for methanol refinement, which advantageously provides a highly efficient and energy-saving solution for producing high-purity methanol. The dividing wall column optimizes the separation process, combining multiple distillation sections into one unit to save energy and reduce operating costs. Additionally, a portion of glycol or glycerol may be recycled, contributing to sustainability and cost-efficiency.

In the detailed description of embodiments of the disclosure above, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a fluid sample” includes reference to one or more of such samples.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

In the description of FIGS. 1-2, any component described with regard to a figure, in various embodiments disclosed herein, may be equivalent to one or more like-named components described with regard to any other figure. For brevity, descriptions of these components are not repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. A process for purifying a methanol stream, comprising:
obtaining a crude methanol feedstock comprising methanol, water, acetone, methyl formate, and optionally one or more of ethanol, propanol, and butanol;
contacting the crude methanol with a glycol, a glycerol, or a mixture of glycols in an azeotropic distillation column to recover a first overhead stream comprising the acetone and methyl formate and a first bottoms stream comprising the glycol or glycerol, water, methanol, and, when contained in the feed, the ethanol, propanol and butanol; and
separating the first bottoms stream in a dividing wall distillation column to recover a second overhead stream comprising methanol and water, a second bottoms stream comprising the glycol or glycerol, a side draw stream comprising water and, when contained in the feed, the ethanol, propanol and butanol.

2. The process of claim 1, wherein the crude methanol feedstock is a crude methanol obtained from a municipal waste-derived or biomass-derived methanol production process.

3. The process of claim 1, wherein the dividing wall distillation column comprises a mid-wall, a common rectification section and a common stripping section respectively located above and below the mid-wall.

4. The process of claim 1, further comprising:
cooling the first overhead stream using a first heat exchanger to produce a first condensed overhead stream;
feeding the first condensed overhead stream to a first reflux drum to produce a first distillate stream;
pressurizing the first distillate stream using a first pump to produce a first pressurized distillate stream;
recycling a first portion of the first pressurized distillate stream to an upper portion of the azeotropic distillation column; and
recovering a second portion of the first pressurized distillate stream as a light impurities stream comprising acetone.

5. The process of claim 1, further comprising:
cooling the second overhead stream using a third heat exchanger to produce a second condensed overhead stream;
feeding the second condensed overhead stream to a second reflux drum to produce a second distillate stream;
pressurizing the second distillate stream using a third pump to produce a second pressurized distillate stream;
recycling a first portion of the second pressurized distillate stream to an upper portion of the dividing wall distillation column; and
recovering a second portion of the second pressurized distillate stream as a purified methanol product.

6. The process of claim 5, wherein the purified methanol product comprises a methanol purity of at least 95 wt. %.

7. The process of claim 1, further comprising recycling the second bottoms stream to the azeotropic distillation column.

8. A process for purifying a methanol stream, comprising:
obtaining a crude methanol feedstock comprising methanol, water, acetone, methyl formate, and optionally one or more of ethanol, propanol, and butanol;
contacting the crude methanol with a glycol, a glycerol, or a mixture of glycols in an azeotropic distillation column to recover a first overhead stream comprising the acetone and methyl formate and a first bottoms stream comprising the glycol or glycerol, water, methanol, and, when contained in the feed, the ethanol, propanol and butanol; and
separating the first bottoms stream in a methanol tailing column to recover a third overhead stream comprising methanol and water, a third bottoms stream comprising the glycol or glycerol, water and, when contained in the feed, the ethanol, propanol and butanol.

9. The process of claim 8, wherein the crude methanol feedstock is a crude methanol obtained from a municipal waste-derived or biomass-derived methanol production process.

10. The process of claim 8, further comprising:
cooling the first overhead stream using a first heat exchanger to produce a first condensed overhead stream;
feeding the first condensed overhead stream to a first reflux drum to produce a first distillate stream;
pressurizing the first distillate stream using a first pump to produce a first pressurized distillate stream;
recycling a first portion of the first pressurized distillate stream to an upper portion of the azeotropic distillation column; and
recovering a second portion of the first pressurized distillate stream as a light impurities stream comprising acetone.

11. The process of claim 8, further comprising:
cooling the third overhead stream using a fifth heat exchanger to produce a third condensed overhead stream;
feeding the third condensed overhead stream to a third reflux drum to produce a third distillate stream;
pressurizing the third distillate stream using a fifth pump to produce a third pressurized distillate stream;
recycling a first portion of the third pressurized distillate stream to an upper portion of the methanol tailing column; and
recovering a second portion of the third pressurized distillate stream as a second purified methanol product.

12. The process of claim 11, wherein the second purified methanol product comprises a methanol purity of at least 95 wt. %.

13. The process of claim 8, further comprising separating the third bottoms stream in a solvent fractionator column to produce a fourth overhead stream and a fourth bottoms stream.

14. The process of claim 13, further comprising:
cooling the fourth overhead stream in a seventh heat exchanger to produce a fourth condensed overhead stream;
feeding the fourth condensed overhead stream to a fourth reflux drum to produce a fourth distillate stream;
pressurizing the fourth distillate stream to produce a fourth pressurized distillate stream;
recycling a first portion of the fourth pressurized distillate stream to an upper portion of the solvent fractionator column; and
recovering a second portion of the fourth pressurized distillate stream as a heavy impurities fraction comprising water, ethanol, propanol, and butanol.

15. The process of claim 13, further comprising:
recycling the fourth bottoms stream to the azeotropic distillation column.

16. A system for purifying a methanol stream, comprising:
a feed stream for supplying a crude methanol feedstock comprising methanol, water, one or more hydrocarbon impurities that form an azeotrope with methanol and one or more hydrocarbons having a boiling point greater than methanol;
an azeotropic distillation column configured for contacting the crude methanol with a glycol or glycerol to recover a first overhead stream comprising the one or more hydrocarbon impurities that form an azeotrope with methanol and a first bottoms
stream comprising the glycol or glycerol, water, methanol, and the one or more hydrocarbons having a boiling point greater than methanol; and
a dividing wall distillation column configured for separating the first bottoms stream to recover a second overheads stream comprising methanol and water, a second bottoms stream comprising the glycol or glycerol, and a side draw stream comprising water and the one or more hydrocarbons having a boiling point greater than methanol.

17. The system of claim 16, wherein the dividing wall distillation column comprises a mid-wall, a common rectification section, and a common stripping section.
